(19) United States
(12) Patent Application Publication
(10) Pub. No.: US 2022/0040375 A1
(43) Pub. Date: Feb. 10, 2022

(54) METHOD OF COATING MEDICAL IMPLANTS WITH HYDROXYAPATITE AND DEVICE FOR IMPLEMENTING THE SAME

(71) Applicant: ARIEL SCIENTIFIC INNOVATIONS LTD., Ariel (IL)

(72) Inventors: Konstantin BORODIANSKY, Petach Tikva (IL); Michael ZINIGRAD, Ramat Gan (IL); Aleksandr SOBOLEV, Ariel (IL); Alexey KOSSENKO, Ashdod (IL); Alexender KRASNOPOLSKI, Petach Tikva (IL)

(21) Appl. No.: 17/422,339

(22) PCT Filed: Jan. 13, 2020
(86) PCT No.: PCT/IL2020/050046

§ 371 Date: Jul. 12, 2021

Related U.S. Application Data
(60) Provisional application No. 62/799,050, filed on Jan. 31, 2019.

(51) Int. Cl. A61L 27/32; C23C 8/36; C23C 8/52

(52) U.S. Cl. CPC A61L 27/32; A61L 2420/02; C23C 8/52; C23C 8/36

(57) ABSTRACT

A method of coating a medical implant with hydroxyapatite comprises steps of: (a) plasma treating said medical implant by a plasma electrolytic oxidation bath within an electrolyte; (b) hydroxyapatite coating a plasma treated medical implant in a hydrothermal pressurized reactor; (c) washing a hydroxyapatite coated medical implant; and (d) drying a washed medical implant. At least one of steps a and b further comprises a sub-step of forming crystallization seeds on a surface of said medical implant.

### FIELD OF THE INVENTION

The present invention relates to coatings applicable to valve metal alloys and, more particularly, to coatings which assist osseointegration of medical implants made of the valve metal alloys within a patient's body.

### BACKGROUND OF THE INVENTION

Orthopedic and dental implants are used routinely worldwide. Those implants indirectly assist in the biological aspects of bone healing by providing stability to bone fractures (S. B. Goodman, Z. Yao, M. Keeney, F. Yang, The future of biologic coatings for orthopaedic implants, Biomaterials 2013, 34 (13), 3174-3183). Ti alloys are mostly used as the main metallic material for implants production. However, coating of HA on those implants is preferable. The biocompatibility of HA was investigated, and it was proved that when HA is presented in the coating, it is spontaneously bonds with living bone (K. de Groot, R. Geesink, C. P. Klein, P. Serekian, Plasma sprayed coatings of hydroxylapatite. J. Biomed. Mater. Res. 1987, 21 (12), 1375-1381; R. G. Geesink, K. de Groot, C. P. Klein, Chemical implant fixation using hydroxyl-apatite coatings. The development of a human total hip prosthesis for chemical fixation to bone using hydroxylapatite coatings on titanium substrates. Clin. Orthop. Relat. Res. 1987, 225, 147-170; Santos-Coquillat, R. Gonzalez Tenorio, M. Mohedano, E. Martinez-Campos, R. Arrabal, E. Matykina, Tailoring of antibacterial and osteogenic properties of Ti6A14V by plasma electrolytic oxidation, Appl Surf Sci 2018, 454, 157-172).

Additionally to osseointegration enhance, HA coatings has a function to seal the interface from wear particles and macrophage associated periprosthetic osteolysis (bone resorption). PEO has been shown as a favorable method for oxide layer formation on Ti alloy which is forward subjected to HA incorporation. HA layer can be deposited on TiO₂ coating by various techniques such as plasma spray, sol-gel methods, electrochemical deposition and electrophoresis (S. Durdu, O. F. Deniz, I. Kutbay, M. Usta, Characterization and formation of hydroxyapatite on Ti6A14V coated by plasma electrolytic oxidation, J. Alloy. Comp. 2013, 551, 422-429; J. Chen, Y. Shi, L. Wang, F. Yan, F. Zhang, Preparation and properties of hydroxyapatite-containing titania coating by micro-arc oxidation, Mater. Lett. 2006, 60 (20), 2538-2543; Z. Q. Yao, Yu. Ivanisenko, T. Diemant, A. Caron, A. Chuvilin, J. Z. Jiang, R. Z. Valiev, M. Qi, H. J. Fecht, Synthesis and properties of hydroxyapatite-containing porous titania coating on ultrafine grained titanium by micro-arc oxidation, Acta Biomater. 2010, 6 (7), 2816-2825).

Unfortunately, all these methods have main disadvantage, delimitation of the HA layer from the titanium alloys due to the weak bonding between the coating and the substrate.

While using PEO, it is shown (A. Sobolev, A. Kossenko, M. Zinigrad, K. Borodianskiy, Comparison of Plasma Electrolytic Oxidation Coatings on Al Alloy Created in Aqueous Solution and Molten Salt Electrolytes, Surf Coat. Technol. 2018, 344, 590-595; A. Sobolev, A. Kossenko, M. Zinigrad, K. Borodianskiy, An Investigation of Oxide Coating Synthesized on an Aluminum Alloy by Plasma Electrolytic Oxidation in Molten Salt, Applied Sciences 2017, 7 (9), 889-898).This works deals with the electrolyte based on molten salt. In our previous works an alternative approach by conducting PEO in molten salt. Using this approach we found that the oxide growth rate is 3 times higher and the energy efficiency is 6 times higher compared to the same process conducted in aqueous electrolyte. In other words, application of proposed technology makes possible to treat much larger surfaces of the implants as used in orthopedic implants or treatment of numerous dental implants in one batch.

Titanium and its alloys have been successfully used as dental and orthopedic biomaterials because of their good mechanical properties, corrosion resistance and biocompatibility with living tissue. However, the bio-inertness of Ti-based implants may inhibit their direct bonding with bone tissue during implantation. Hence, efforts have been made to strengthen the bonding between implant and bone tissue to enhance osseointegration and reduce the healing period mostly, using hydroxyapatite (HA). The term osseointegration refers to the structural and functional connection between bone cells and the surface of an artificial implant and was discovered in 1952 by the Swedish professor Per-Ingvar Brånemark who found that titanium attaches itself to bone when it is implanted in it.

HA can be applied on the Ti surface by conventionally adopted methods such as thermal spraying, sputter coating, hot pressing, acid-etching and plasma spraying. Unfortunately, all these methods have disadvantage, delimitation of the HA layer from the surface due to the poor bonding between the coating and the substrate.

Plasma Electrolytic Oxidation (PEO) followed by special hydrothermal treatment process is an alternative approach to obtain HA on Ti implant surface. HA is formed inside the layer of titanium oxide, so that the coating forms much stronger bonds with the substrate.

PEO process usually carried out in aqueous electrolyte which main disadvantages are the necessity of the forced cooling of the electrolyte bath, a high current density, the thermal dissociation of the electrolyte and the formation of mixed compounds in the ceramic coating and coating low growing rate. These issues can be solved by the replacing an aqueous electrolyte by molten salts.

### SUMMARY OF THE INVENTION

It is hence one object of the invention to disclose a method of coating a medical implant with hydroxyapatite. The aforesaid method comprises steps of: (a) plasma treating said medical implant by a plasma electrolytic oxidation bath within an electrolyte; (b) hydroxyapatite coating a plasma treated medical implant in a hydrothermal pressurized reactor; (c) washing a hydroxyapatite coated medical implant; and (d) drying a washed medical implant.

It is a core purpose of the invention to provide at least one of the steps a and b further comprising a sub-step of forming crystallization seeds on a surface of said medical implant.

Another object of the invention is to disclose the electrolyte comprising a molten salt.

A further object of the invention is to disclose the molten salt said comprising a salt selected from the group consisting of nitrate, carbonate, sulfate, silicate, chloride and any combination thereof.

A further object of the invention is to disclose the molten salt which is doped with calcium.

A further object of the invention is to disclose the molten salt comprising calcium dichloride.

A further object of the invention is to disclose the molten salt which is doped with phosphorus.

A further object of the invention is to disclose the molten salt comprising trisodium phosphate.

A further object of the invention is to disclose the step of hydroxyapatite coating comprising treating said medical implant in aqueous solution of potassium hydroxide.

A further object of the invention is to disclose the step of hydroxyapatite coating comprising treating said medical implant in aqueous solution of ammonium phosphate.

A further object of the invention is to disclose the medical implant made of an alloy comprising metals selected from the group consisting of Aluminum, Tantalum, Niobium, Zirconium, Titanium, Bismuth, Stibium, Magnesium, Zink, Cadmium, Tungsten, Stannum, Iron, Silver, Hafnium, Beryllium, Germanium, Silicon, Uranium and any combination thereof.

A further object of the invention is to disclose a device for coating a medical implant with hydroxyapatite. The aforesaid device comprises: (a) a plasma electrolytic oxidation bath accommodating an electrolyte; and (b) a hydrothermal pressurized reactor accommodating an alkaline solution.

A further object of the invention is to disclose at least one of processing media accommodated in said plasma electrolytic oxidation bath and hydrothermal pressurized reactor contain precursors assisting formation of crystallization seeds on a surface of said medical implant.

### BRIEF DESCRIPTION OF THE DRAWINGS

In order to understand the invention and to see how it may be implemented in practice, a plurality of embodiments is adapted to now be described, by way of non-limiting example only, with reference to the accompanying drawings, in which

FIG. 1 is a schematic view of a device for coating a medical implant made of an alloy containing a valve metal with hydroxyapatite; and

FIG. 2 is a flowchart of a method of coating a medical implant made of an alloy containing a valve metal with hydroxyapatite.

### DETAILED DESCRIPTION OF THE INVENTION

The following description is provided, so as to enable any person skilled in the art to make use of said invention and sets forth the best modes contemplated by the inventor of carrying out this invention. Various modifications, however, are adapted to remain apparent to those skilled in the art, since the generic principles of the present invention have been defined specifically to provide a method of coating a medical implant made of an alloy containing a valve metal with hydroxyapatite and a device for implementing the same.

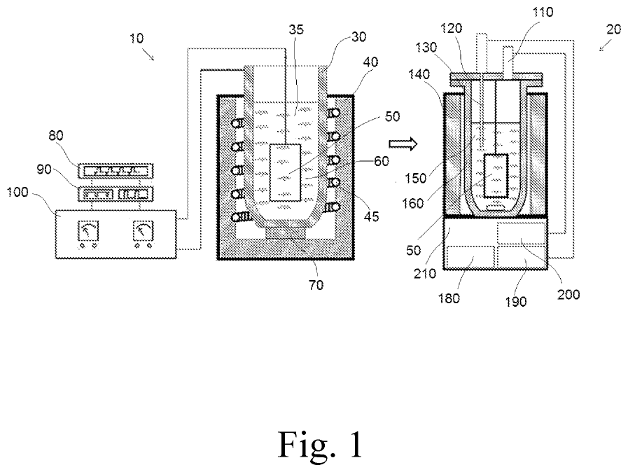

Reference is now made to FIG. 1 presenting a schematic view of a device for coating a medical implant made of an alloy containing a valve metal with hydroxyapatite. The device comprises plasma electrolytic oxidation (PEO) bath 10 and hydrothermal treatment reactor 20. In PEO bath 10, numeral 40 refers to a furnace with automatic temperature control. The furnace includes heating element 45 energized by power supply 100 which is configured for heating metallic crucible 30 made of a nickel alloy. Crucible 30 accommodates electrolyte 35 comprising molten salts. Crucible 30 is supported by ceramic stand 70. Article 50 made of an alloy containing a valve metal is oxidized within molten salts such that PEO treatment is performed. Numerals 80 and 90 refer to a data logger and a pulse generator, respectively.

According to an exemplary embodiment of the present invention, article 50 made of a titanium alloy is treated in PEO bath accommodating molten KNO₃ and NaNO₃. Other salts selected from the group consisting of nitrate, carbonate, sulfate, silicate, chloride are also in the scope of the present invention. The following parameters of the PEO treatment are feasible:
Temperature in PEO bath: ±300° C. form the melting point of the electrolyte;
Current density: 0.05-100 A/dm²;
Voltage: 10-500V;
Mode: potentiostatic or potentiodynamic;
Polarity: DC (unipolar or bipolar); AC (symmetric or asymmetric); impulse (unipolar or bipolar);
Impulse sweep: square-wave, trapezoid, sinusoidal, etc.;
Frequency: 0-5000 Hz;
Duty cycle: 1-100%.

The purpose of the present invention is to form crystallization seeds on a surface of treated article 50. According to one embodiment of the present invention, Ca and P precursors can introduced into electrolyte 35 (for example, calcium dichloride and trisodium phosphate). Then, article 50 is transferred into hydrothermal treatment reactor 20 filled with alkaline solution (for example, potassium hydroxide).

Hydrothermal treatment reactor 20 comprises stirring-heating plate 210 including control units 180, 190 and 200 configured for controlling stirring speed, temperature and pressure and, respectively. Unit 200 is configured for receiving data from pressure gauge 110, unit 190 from thermocouple 130. Stainless steel crucible 160 is placed into isolating chamber 140. Crucible 160 is filled with alkaline solution 150.

According to one embodiment of the present invention, calcium and phosphorus precursors are formed into electrolyte 35.

The hydrothermal treatment is carried out in pressurized reactor 20 in aqueous solution of KOH for 2 h at 200° C., 16atm and pH=11. Finally, Ti based alloy with hydroxyapatite-based coating with the high surface area is formed.

According to an alternative embodiment of the present invention, calcium precursors are formed in PEO electrolyte 35 by introducing calcium dichloride, while potassium hydroxide solution is replaced with an aqueous solution of ammonium phosphate in order to form phosphorous precursors.

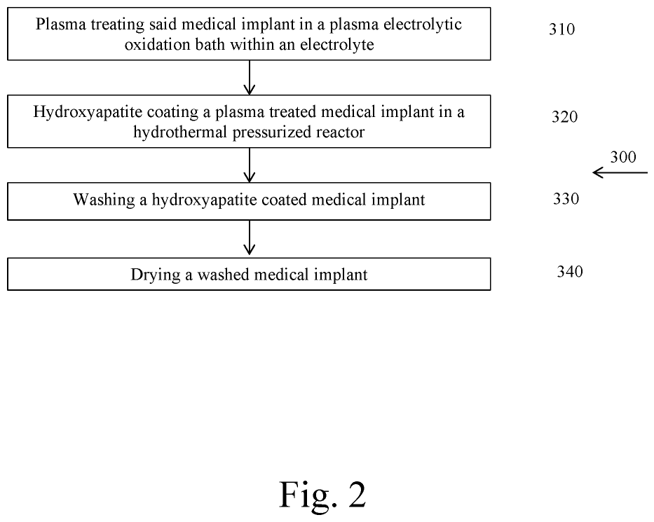

Reference is now made to FIG. 2 presenting a flowchart of method 300 of for coating a medical implant made of an alloy containing a valve metal with hydroxyapatite. An article made of an alloy containing a valve metal is treated in electrolyte of plasma electrolytic oxidation bath at step 310. Then, the article is coated with hydroxyapatite in a hydrothermal treatment reactor at step 320. The treated article is washed and dried at steps 330 and 340, respectively.

It should be emphasized that method 300 is specifically directed to producing coatings on valve metal alloys adapted for osseointegration of medical implants when installed.

Preferred embodiments of this invention are described herein, including the best mode known to the inventors for carrying out the invention. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1-20 (canceled)

21. A method of coating a medical implant with hydroxyapatite; said method comprising steps of:
a. plasma treating said medical implant by a plasma electrolytic oxidation bath within an electrolyte;
b. hydroxyapatite coating a plasma treated medical implant in ahydrothermal pressurized reactor;
c. washing a hydroxyapatite coated medical implant; and
d. drying a washed medical implant;
wherein said electrolyte comprises a molten salt doped with precursors of Calcium and Phosphorus such that crystallization seeds on a surface of said medical implant are formed.

22. The method according to claim 21, wherein said molten salt said comprises a salt selected from the group consisting of nitrate, carbonate, sulfate, silicate, chloride and any combination thereof.

23. The method according to claim 21, wherein said molten salt is doped with calcium.

24. The method according to claim 23, wherein said molten salt comprises calcium dichloride.

25. The method according to claim 21, wherein said molten salt is doped with phosphorus.

26. The method according to claim 25, wherein said molten salt comprises trisodium phosphate.

27. The method according to claim 21, wherein said step of hydroxyapatite coating comprises treating said medical implant in aqueous solution of potassium hydroxide.

28. The method according to claim 21, wherein said step of hydroxyapatite coating comprises treating said medical implant in aqueous solution of ammonium phosphate.

29. The method according to claim 21, wherein said medical implant is made of an alloy comprising metals selected from the group consisting of Aluminum, Tantalum, Niobium, Zirconium, Titanium, Bismuth, Stibium, Magnesium, Zink, Cadmium, Tungsten, Stannum, Iron, Silver, Hafnium, Beryllium, Germanium, Silicon, Uranium and any combination thereof.

30. A device for coating a medical implant with hydroxyapatite; said device comprising:
a. a plasma electrolytic oxidation bath accommodating an electrolyte; and
b. a hydrothermal pressurized reactor accommodating an alkaline solution;
wherein at least one of plasma electrolytic oxidation bath comprising comprises a molten salt doped with precursors of Calcium and Phosphorus such that crystallization seeds on a surface of said medical implant are formed.

31. The device according to claim 30, wherein said molten salt said comprises a salt selected from the group consisting of nitrate, carbonate, sulfate, silicate, chloride and any combination thereof.

32. The device according to claim 30, wherein said molten salt is doped with calcium.

33. The device according to claim 32, wherein said molten salt comprises calcium dichloride.

34. The device according to claim 30, wherein said molten salt is doped with phosphorus.

35. The device according to claim 34, wherein said molten salt comprises trisodium phosphate.

36. The device according to claim 30, wherein said alkaline solution is an aqueous solution of potassium hydroxide.

37. The device according to claim 30, wherein said alkaline solution is an aqueous solution of ammonium phosphate.

38. The device according to claim 30, wherein said medical implant is made of an alloy comprising metals selected from the group consisting of Aluminum, Tantalum, Niobium, Zirconium, Titanium, Bismuth, Stibium, Magnesium, Zink, Cadmium, Tungsten, Stannum, Iron, Silver, Hafnium, Beryllium, Germanium, Silicon, Uranium and any combination thereof.
